(19) United States
(12) Patent Application Publication
(10) Pub. No.: US 2014/0303447 A1
(43) Pub. Date: Oct. 9, 2014

(54) SURGICAL DRAPE SYSTEM FOR UROLOGY PROCEDURES ON FEMALE PATIENTS

(71) Applicants: Errol O. SINGH, Columbus, OH (US); Ake A. HELLSTROM, Columbus, OH (US); Walter RUSSELL, Westerville, OH (US)

(72) Inventors: Errol O. SINGH, Columbus, OH (US); Ake A. HELLSTROM, Columbus, OH (US); Walter RUSSELL, Westerville, OH (US)

(73) Assignee: OHIO UROLOGIC RESEARCH, LLC, Columbus, OH (US)

(21) Appl. No.: 14/287,545

(22) Filed: May 27, 2014

Related U.S. Application Data
(63) Continuation of application No. 14/111,983, now abandoned.
(60) Provisional application No. 61/473,150, filed on Apr. 8, 2011.

(51) Int. Cl. A61B 19/08; A61B 1/307; A61B 1/00; A61B 1/07; A61B 17/02; A61B 1/32; A61B 1/06

(52) U.S. Cl. CPC A61B 19/087; A61B 1/32; A61B 1/307; A61B 1/0684; A61B 1/07; A61B 17/0293; A61B 1/00082; A61B 2017/0287; A61B 2019/085
USPC 600/207; 600/235; 600/212; 600/208

(57) ABSTRACT

The invention is a simple surgical drape system for female urology procedures that provides sealed isolation of top versus bottom sides of the drape and only exposes the female urethral meatus area for the procedure. It is achieved by a combination of a drape with a fenestration, a labia spreader where the drape fenestration is attached to provide a seal, and local illumination of the meatus area integrated in the drape system for safer and easier instrument insertion. The labia spreader also frees up one of the operator's hands and provides protection for false catherization. The labia spreader can have a vaginal inserted portion for precise position fixation of the drape fenestration to the female. The drape system also provides improved liquid management made possible by the unique combination of drape and labia spreader acting as seal between the two sides of the drape.

### CROSS-REFERENCE TO RELATED APPLICATIONS

This application claims benefit of U.S. Provisional Application 61/473,150, filed Apr. 8, 2011.

### BACKGROUND OF THE INVENTION

This invention is in the technical field of medical equipment, in particular for surgical drape systems utilized in urology procedures on female patients. Such drapes have up to now typically been of unisex design, introducing mis-fit and non sterile conditions due to the special female anatomy. Such procedures are relatively common but still have demonstrated less than desired outcome in many cases. Problems may arise with infections and patient discomfort that require prolonged patient care and procedure rework in best cases, but can in worse cases introduce serious long term consequences both for patients and care givers. This occurs currently despite more stringent medical guidelines being in place. There is a very active demand today by the entire society to reduce health care cost, and medical procedure infections and other procedure complications is one important factor.

A particular problem in urology procedures on female patients is the difficulty to properly insert catheters, endoscopes or other instruments in the urethral meatus. Typically, one hand of the operator must be dedicated to provide access by spreading the labia during most of the procedure. This impairs the operator to perform other important tasks with the patient or with instruments. It also increases infection risk by touching contaminated or loose particulate areas and spreading this to the urethral meatus or to instruments.

As another problem, the female anatomy also makes the urethral meatus area difficult to clearly observe with normal overhead illumination, due to the meatus recessed location or shadows from operator hands, instruments or drapes. This is compounded by the operator having only one hand free, while the other must spread the labia. Therefore, quite often mis-insertion will occur, with the instrument entering or touching the labia or vaginal vault instead of entering the urethra. This causes a risk for the instrument being contaminated by vaginal bacteria flora. It will in best case result in the operator discarding the instrument and picking a new sterile unit for a re-try. But due to time press and need for medical cost containment, or just difficulty to clearly view the female urethral meatus area, the same contaminated instrument may enter the urethra in a re-try. This could cause urinary tract infections or other procedure complications.

Additionally, as part of preparation for urology procedures like catheterization, the patient is prepared with antibacterial agents in the pubic area and nearby body parts. This process has the complication that this particular area is typically partially covered with body hair and has several skin folds, resulting in risk for incomplete bacteria elimination. Furthermore, loose hair and skin particles from this area may easily become dislodged during the urology procedure and can contaminate the operator's gloves, urology instruments, or the urethral meatus and nearby areas. This may call for using extra potent antibacterial agents. However, increasing the amount, or potency, of antibacterial agents can lead to the long term drawback of breeding more resistant bacteria strains.

In a female urology procedure, the patient is typically cleaned and prepared, and then covered partially with one or more sterile surgical drapes. The drape may use separate sections or have one or more fenestrations included to provide access to the female organ. It is common that sizeable amounts of liquids emerge from the urethra or instruments during the procedure. This can cause patient wetness, discomfort, infectious matter spreading, or inconvenience for the operator. Drape fenestrations or operator shields have in prior art been attempted to control liquid flow. Such liquids may still flow down behind fenestration apertures and reach other parts of the patient body and the operating table, causing inconvenience, sterile area contamination and a generally less clean environment. Medical spent liquids disposal needs special procedures and equipment to avoid spills or undesired contact.

The female genital anatomy makes it difficult to hold surgical drapes tightly in place in near contact with the body. Methods and devices are known for holding drapes tightly to the body in medical procedures for other body areas include straps, medical tape, temporary adhesives, and alike. However, these are difficult to implement effectively on the female genitals, due to the concave nature of the anatomy, the sensitive tissue, and presence of body hair.

These drawbacks have been attempted to be solved to varying degree of success by prior art, but there is to our knowledge not any solutions disclosed that solves the combination of all these issues with one surgical drape system, yet minimum drawbacks for the urology procedure, and coupled with design and user simplicity, and procedure economy.

### SUMMARY OF THE INVENTION

The present invention is a drape system for reduced risk and simplified urological procedures for female patients. The drape system includes a subset, or all, of the following main components:
1) A drape with a fenestration providing urethral meatus access,
2) A labia spreader that also holds the drape in place and sealed onto the patient,
3) Integrated local illumination to visualize the female urethral meatus
4) A liquid absorbing pad and a liquid control flap.

The novel drape system provides the following benefits—simultaneously or separately:
1) Isolation of urethral meatus access from other body parts, for reduced infection risk
2) An integrated labia spreading device, freeing up one hand for the operator
3) Easier and safer catheterization or other instrument entry due to integrated illumination
4) Drape can be attached to labia spreader for sealing and stability of drape
5) A vaginal insertable portion of the labia spreader for keeping drape and urethral access fenestration tightly in place at a controlled position
6) A barrier reducing the probability of mis-catherization into the vaginal tract
7) A quick-assembly method of the drape system by adhesive surface
8) Urology liquid discharges stay on the outside of the drape, making liquid management easier, less risk for contamination, and patient more comfortable
9) Urology liquid management for protection and ease of medical waste disposal
10) With catheters or similar urology instruments still in the patient, the entire drape system is still easily removed

The drape system separates a non-sterile environment on the patient side of the drape and a sterile environment on the operator side of the drape, with a sealed fenestration between these areas. This prevents contamination from other body parts to reach the operator side, and reducing risk of urology fluids on the operator side to reach the patient side. The exposed patient area on the top sterile side is limited to only the urethral meatus with near surroundings, and provides a seal from the non sterile areas of the patient. This improves outcome of antibacterial preparation of the patient ahead of the urological procedure, and reduces infection risk by isolating the urology procedure location from the pubic area.

Because this drape design creates a sealed area on the operator side of the patient except the meatus area, control and collection of urology liquids is simplified. For instance, a disposable and safe to handle absorbing or liquid gelling pad may be placed on portions of drape operator side and a drape flap may control flows.

Removal of the drape after the urology procedure with instruments like catheters still in place can be achieved by a splittable line in the drape all the way in from an external drape edge and in to the fenestration. The other components in the drape system are designed in such a way that an inserted catheter cannot capture any device of the system.

As a summary, this female patient urology drape system offers major advantages in safety, functionality, simplicity, predictable procedures and total cost versus prior art.

Thus, in accordance with the present invention, there is provided a medical system for female urological procedures, the system including a labia spreading device allowing access to the urethral meatus, a flat, flexible, sterile drape for overlaying the spreading device and having an aperture to be located over the access opening of the spreading device, and preferably also one or more illumination means for illuminating the urethral meatus, the illumination means being carried by the spreading device or the drape when the system is in use.

In one embodiment of the invention, the illumination means comprises a light emitting diode powered by a battery.

In another embodiment of the invention, the illumination means is integrated into the spreading device or into the drape.

In certain embodiments of the invention, the light source is carried on the exterior of the spreading device or the drape, while in other inventive embodiments, the light source is carried in the interior of the spreading device or the drape.

In other embodiments of the invention, the spreading device and the drape are adhesively attached to each other during use.

In other embodiments of the invention, new labia spreading devices are disclosed, some of which carry one or more light sources

In still another embodiment of the invention, there is provided a medical method for providing a sterile environment during a female urological procedure, which method comprises the use of a medical system of the present invention, one aspect of which can include the external use of a liquid absorbing pad in combination with a liquid control foldable flap attached to the exterior of the drape.

### BRIEF DESCRIPTION OF THE DRAWINGS

FIG. 1 shows a representation of antibacterial cleaning in prior art.

FIG. 2 shows a urological procedure in prior art

FIG. 3 shows a simplified planar view of the drape of the invention

FIG. 4 shows a planar view of the drape of including a different material insert

FIG. 5 shows an enlarged planar view of the fenestration in the drape

FIG. 6 shows a cross section of the fenestration

FIG. 7 shows an example of the labia spreading device, viewed from the drape side

FIG. 8 shows a vertical cross section of a labia spreading device

FIG. 9 show a top view of a labia spreading device

FIG. 10 shows a perspective view of a labia spreading device

FIG. 11 shows an alternate design of the flange in the labia spreading device

FIG. 12 shows a flexible labia spreading device deformed for insertion

FIG. 13 shows an alternate labia spreading device with split flange

FIG. 14 shows an alternate labia spreading device with U-form.

FIG. 15a and FIG. 15b show the U-form labia spreading device spring action

FIGS. 16, 17 and 18 show different views of a labia spreader with direct illumination

FIGS. 19, 20 and 21 show different views of a labia spreader with light guide illumination

FIG. 22 shows a U-form labia spreader with illumination

FIGS. 23a and 23b show a labia spreader with self adhesive for drape and peel-off cover

FIG. 24 shows attachment of the drape to a labia spreader

FIG. 25 shows attachment of the drape to a U-shape labia spreader

FIG. 26 shows a cross section of the drape attached to a labia spreader

FIG. 27 shows the operator's view of the drape system in place on a female patient

FIG. 28 shows a vertical cross section of the drape system in place on a female patient.

FIG. 29 shows a variation in embodiment with an illumination device attached to the drape.

FIG. 30 shows a variation in embodiment with a plug-in illumination device.

### DETAILED DESCRIPTION OF THE INVENTION

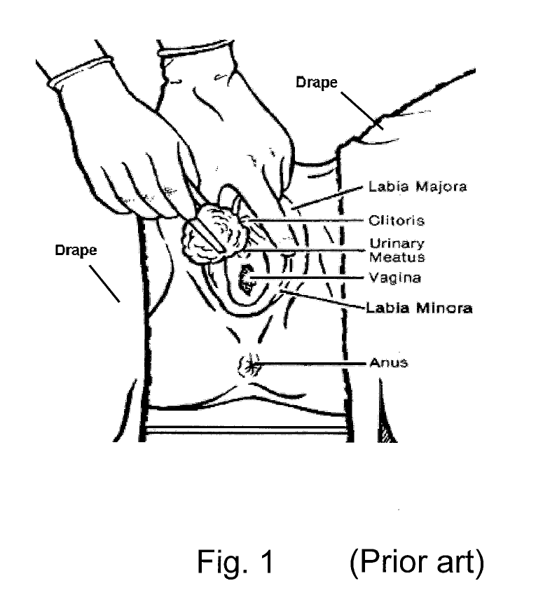

The purpose of the invention is easiest clarified by first a brief comparison to prior art. FIG. 1 (prior art) shows a female patient being prepared by antibacterial swab before a urological procedure, according to prior art. The whole exposed genital area must be treated. It is noted that exposed body hair and skin folds will create crevices that may harbor particles and bacteria that is difficult to fully reach with the swab.

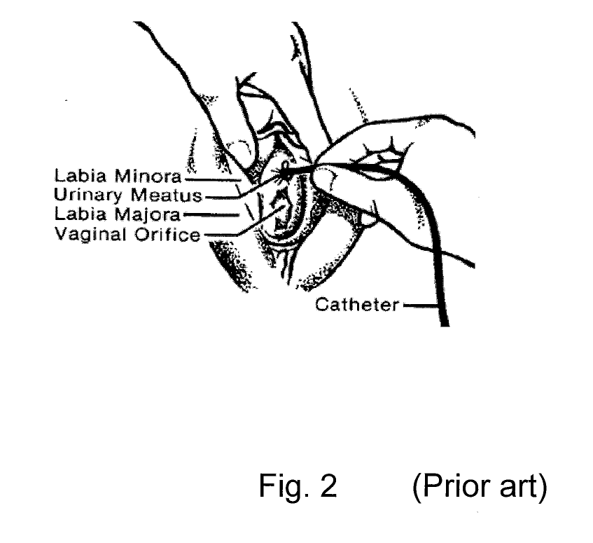

FIG. 2 (prior art) shows a female catherization or similar urology procedure performed according to prior art. It is noted that hair or particles in the genital area may become dislodged during the process and reach the urethral meatus, or the operator's sterile gloves, or insertable urology instruments. It is also noted that labia must be supported by two fingers of one hand during the process, or labia will obscure the meatus access. Furthermore, the need for one hand providing support during most of the process makes it inconvenient for the operator to do other duties during the procedure. It is also noted that due to the open path to the pubic area, liquids emerging from the patient or from treatment can flow down onto the patient and operating table.

The system of the present invention comprises at least a labia spreader and a drape, and preferably also includes a light source (source of illumination or illumination means) carried by the spreading device or by the drape.

By the term “system’ as used herein, is meant that the individual components of the system are brought together at least by the time of use of the components in a female urological procedure. Thus, when the system contains two components of a labia spreader and drape, these two components can be jointly packaged or separately packed in separate containers, prior to use. The two components can be adhesively combined prior to use or at the time of use. When the system includes three components (the third component is the light source), two of them or all of them can be packaged as a connected unit in which the three components are in someway attached to one another, or in a bi-connected unit in which two components are attached to one another, or the three units can be separately packaged. Often, the light source is attached to or forms part of the spreading device or of the drape. The three components, in certain inventive embodiments, are attached to each other at the time of use.

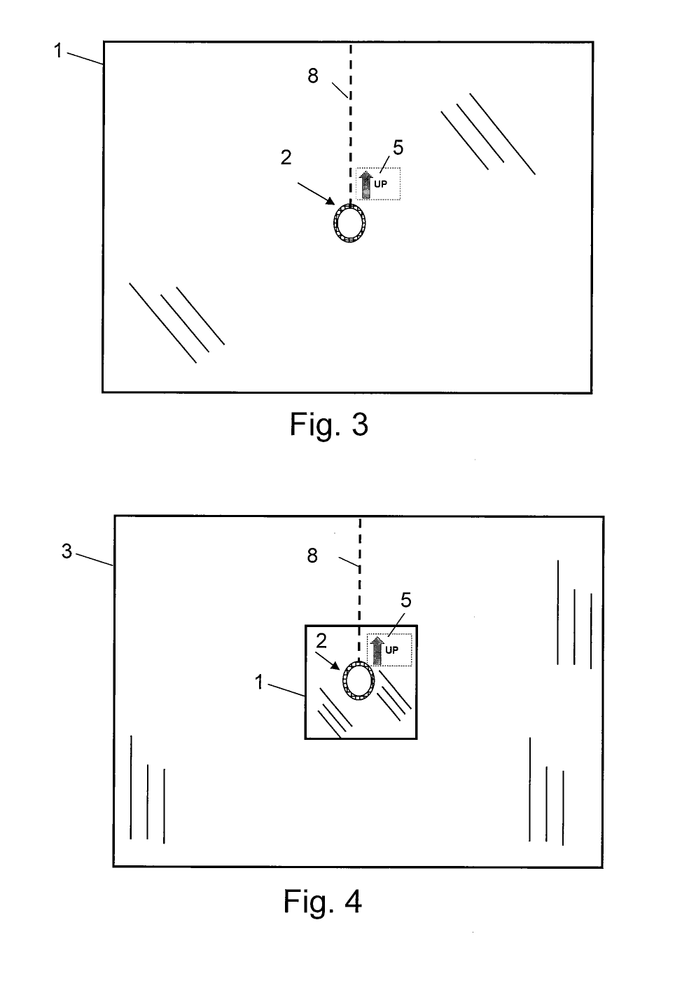

The invention drape is shown in FIG. 3 and includes a drape 1 with a fenestration 2 and a split line 8. It may also include an orientation mark with printed instructions 5. The drape 1 may be made of a transparent material, for instance clear plastics, or by a thin elastic film, for instance latex. The plastics used in making the drape are well known in the prior art and, for example, include vinyl (such as polyvinyl chloride), polyethylene, polypropylene, polycarbonate, polyester, silicone elastomer, acetate and so forth film materials. As an example, the drape may be 1 to 5 mil (0.025-0.125 mm) in thickness. The purpose of the fenestration split line is to enable drape removal with catheters in place. The shape of the drape can be planar and rectangular as shown, but may also be cut to any shape or formed to fit the female anatomy and include additional drape shape features known from prior art.

In FIG. 4 is shown a variation of the design where the drape 1 is attached to a surrounding larger drape sheet 3 that is made of a different material, for instance textile, non-wovens, or opaque plastics.

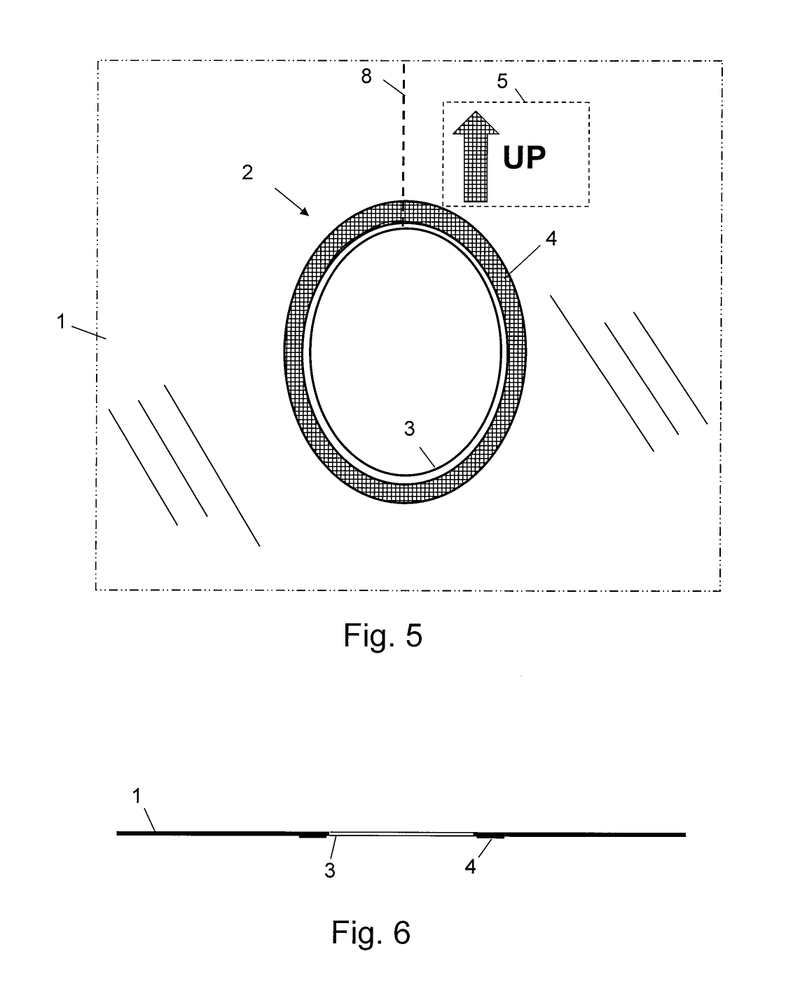

FIG. 5 shows an example of fenestration details. The aperture 3 may be surrounded with a marking 4 on the drape 1 in order to enable operator to make a quick location of the fenestration, in particular useful on transparent drape materials to save time. The marking 4 may be printed or a separate part attached to the drape, where it may also act as a gasket material or adhesive for attachment to the labia spreader.

FIG. 6 is a cross section through the aperture 3. It may simply be a cut hole in the drape or it may have edge reinforcements if material is very thin.

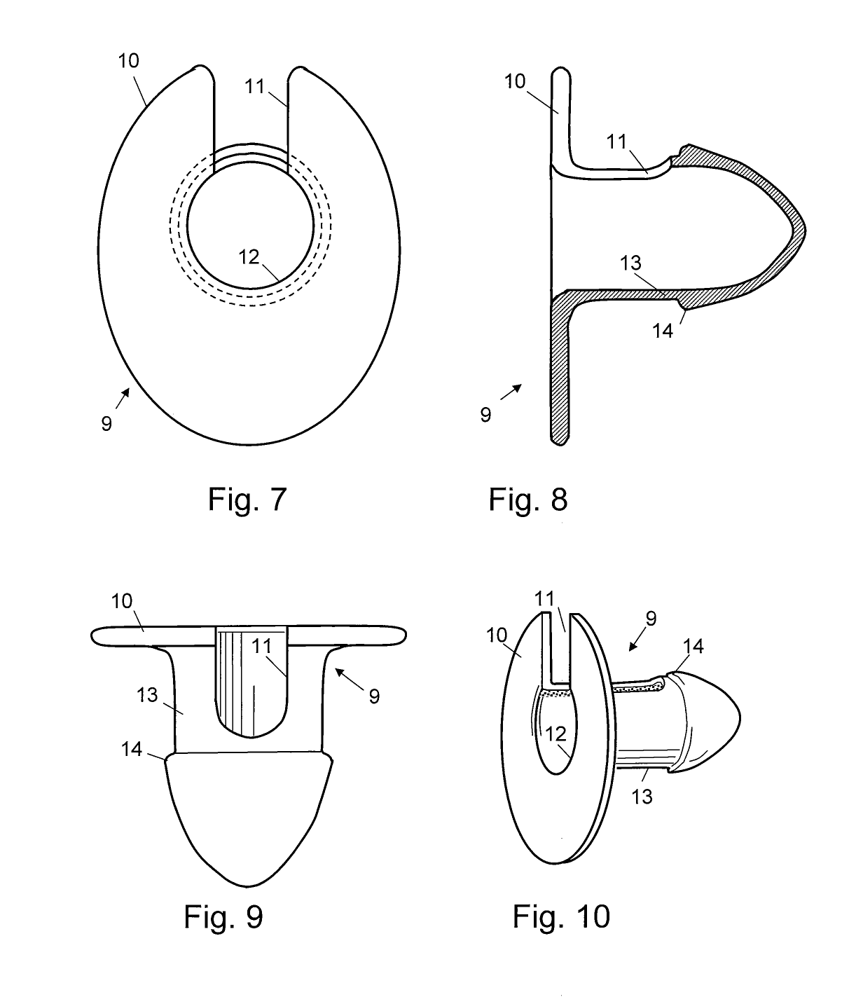

FIG. 7 shows a view from the drape side on one of many possible embodiments for the labia spreader 9. In this design, the labia are spread by the flange 9 that includes a slot 11 and an aperture 12 in order to provide access to urethral meatus. The slot allows removal of the labia spreader with a catheter in place. In this example, the shape of the flange 9 is elliptical, however many other shapes like polygons or circular may be utilized without deviating from the spirit of the invention.

In FIG. 8 is shown a vertical cross section of the same embodiment of labia spreader 9, FIG. 9 shows a view from the top side and FIG. 10 shows a perspective view. The flange 10 is attached to the body or shaft 13, or preferably both made as one molded part. The body or shaft 13 is inserted in the vagina and includes a closed end to prevent false catherization. It is remarked that the slot 11 in the flange 10 as shown may continue a portion into the body 13. This is to permit access to the urethral meatus despite personal anatomy variations. The body 13 may furthermore include a locally widening section 14 forming an enlarged or bulbous head to better secure the labia spreader 9 in inserted position. In the shown embodiment this is molded in shape, but it may also be achieved by other means, for instance a balloon that can be inflated from outside of the patient. In one embodiment, shaft 13 has an annular cross-section and the urethral meatus opening is formed between an outer radius of the shaft and the periphery of flange 10.

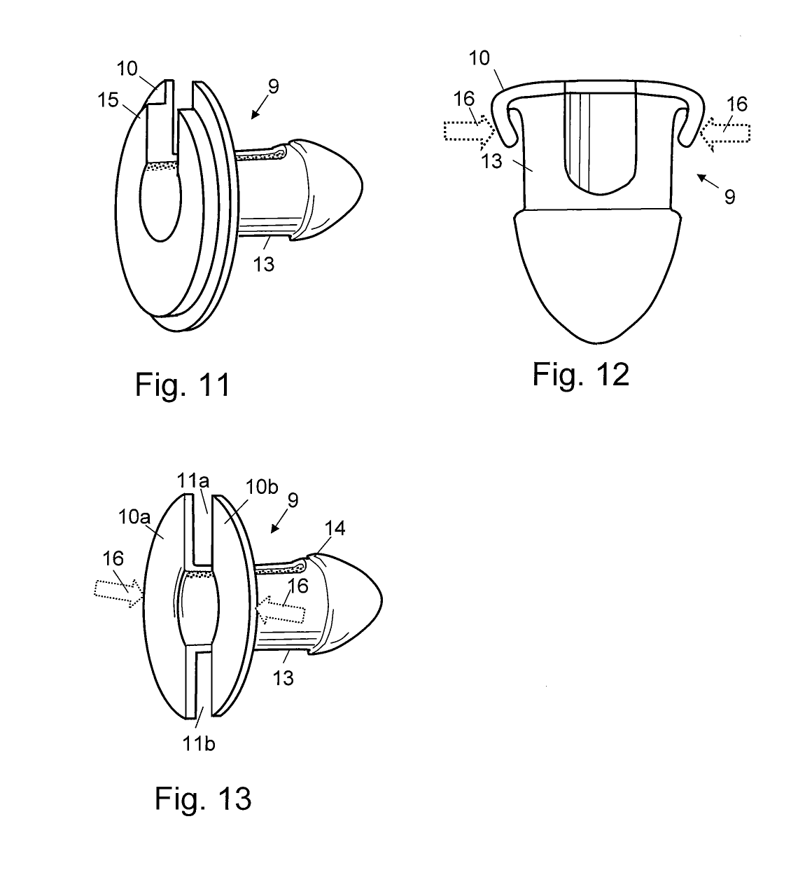

FIG. 11 show a perspective view of a labia spreader 9 with a different cross section shape of flange 10 with the protruding central flange surface 15. The purpose of this is to enable the flange 10 to be positioned and held between the spread labia, while the protruding surface 15 extends beyond the labia, to easier facilitate attachment to the drape to the labia spreader flange 10. The illustration shows sharp corners just for ease of drawing, but for patient comfort, all corners on the labia spreader 9 may have generous radii.

Now going to FIG. 12, this shows a variation in the embodiment where the labia spreader 9 is molded in a flexible material to permit easier insertion. In this example, the operator bends the flange 10 by finger force 16 to a smaller footprint and inserts between labia. Upon release, the flange 10 will expand to normal shape and grip the labia in spread position. Making the flange 10 of a flexible rubber-like material will promote single handed insertion. The entire labia spreader 9 may be molded in a flexible material.

FIG. 13 shows an alternate design for the labia spreader 9 and utilizing flexible materials with spring property. By including two slots 11a and 11b, the flange 10 gets divided into two parts 10a and 10b. When operator applies finger force 16, the flanges 10a and 10b can be held closer to ease insertion in the patient and then allowed to expand by spring force for secure grip. The spring force may originate in the flexible material of labia spreader 9, or it may also include separate springs known from prior art. The labia spreader 9 may also be pre-packaged in a folded-out shape in order to increase the separation force between the flanges 10 a and 10b.

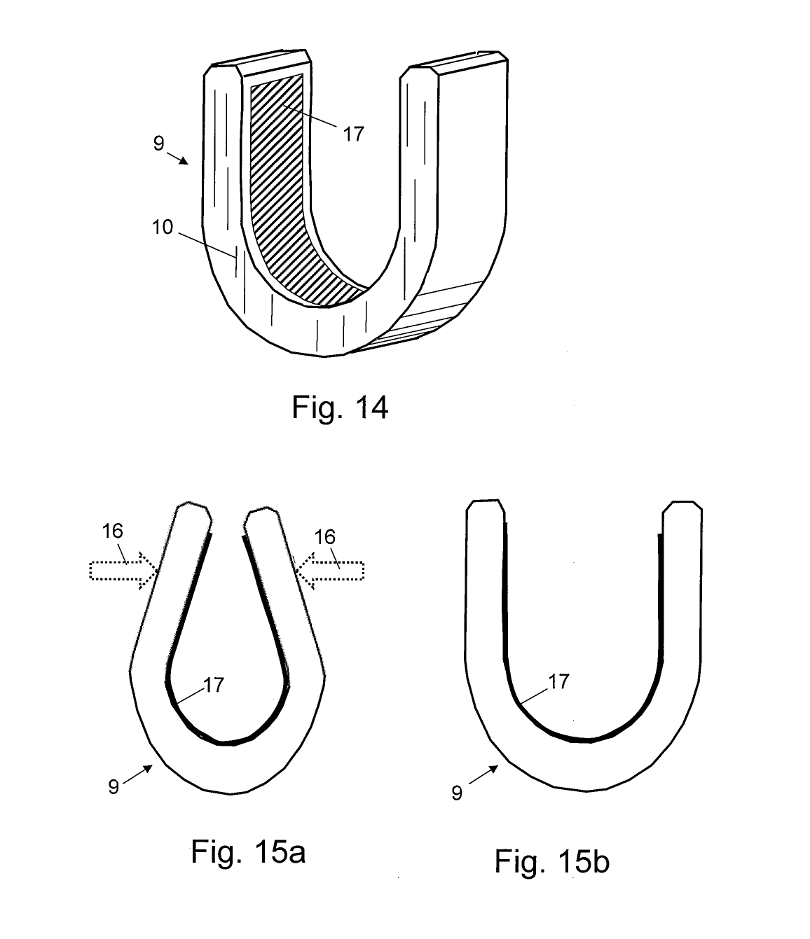

FIGS. 14, 15a and 15b show yet another variation on a labia spreader utilizing flexible materials. In FIG. 14, it is shown an U-shaped labia spreader 9 which preferably is made of a soft material. The material may have inherent spring properties, or be aided by a built in spring member 17 while the rest of the material may be soft. One of the sides 10 is generally flat and will serve as attachment flange to the drape, as will be shown in later illustrations. The opposing side may be flat or contoured for vaginal entry. In FIGS. 15a and 15b is illustrated how the labia spreader is pinched together by finger force 16 before insertion and then expands in place, thus separating the labia.

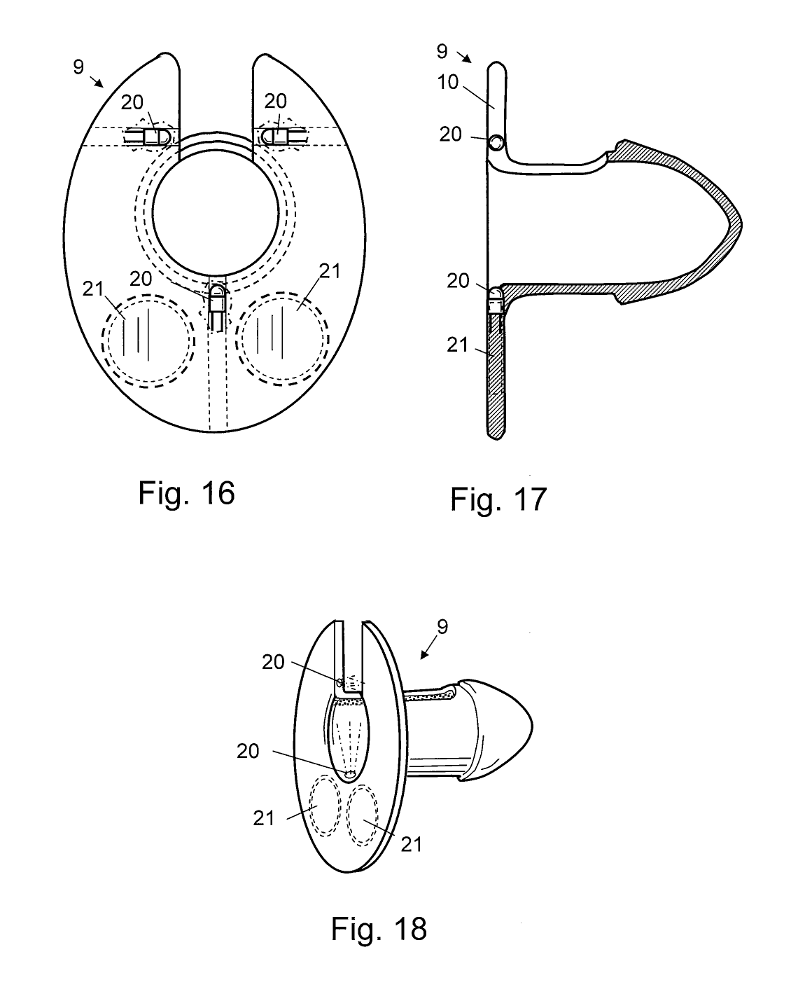

Now going to FIGS. 16, 17 and 18, theses show different views of optional illumination devices included in the labia spreader. The purpose of this illumination is to shine light onto the urethral meatus area, to make catheter insertion clearly visible for the operator and improve success at first try. Note that the typical general clinical room illumination from the ceiling is partially shadowed by the female anatomy and operator's hand to separate the labia. The illumination light intensity can easily be made ample with small power due to the closeness of illuminator to the meatus target. In this example, light is generated by LEDs (light emitting diodes) 20 that can be one or several, and they may be mounted directly into the labia spreader 9. For instance, the flange 10 may hold the LEDs 20 as well as batteries 21. The illumination does not need to be engaged very long time for catheter entry, so relatively small size batteries will suffice for a urology procedure. The LEDs 20, the batteries 21 and associated wiring and other electrical components may be overmolded in to the labia spreader 9, or fitted in cavities that are sealed after the illumination components are inserted. Yet another way of fabricating the labia spreader with included illumination is to make the labia spreader 9 as a two-part design wherein the illumination components are mounted in open cavities before joining the labia spreader parts to create a one-piece sealed unit.

It is pointed out that while FIGS. 16-18 shows light sources and batteries are internal to the material of labia spreader 9, variations of embodiments of the invention include light sources or batteries mounted on exterior of labia spreader 9 or on holders that are part of labia spreader 9, or utilize remote electric power source instead of integrated batteries.

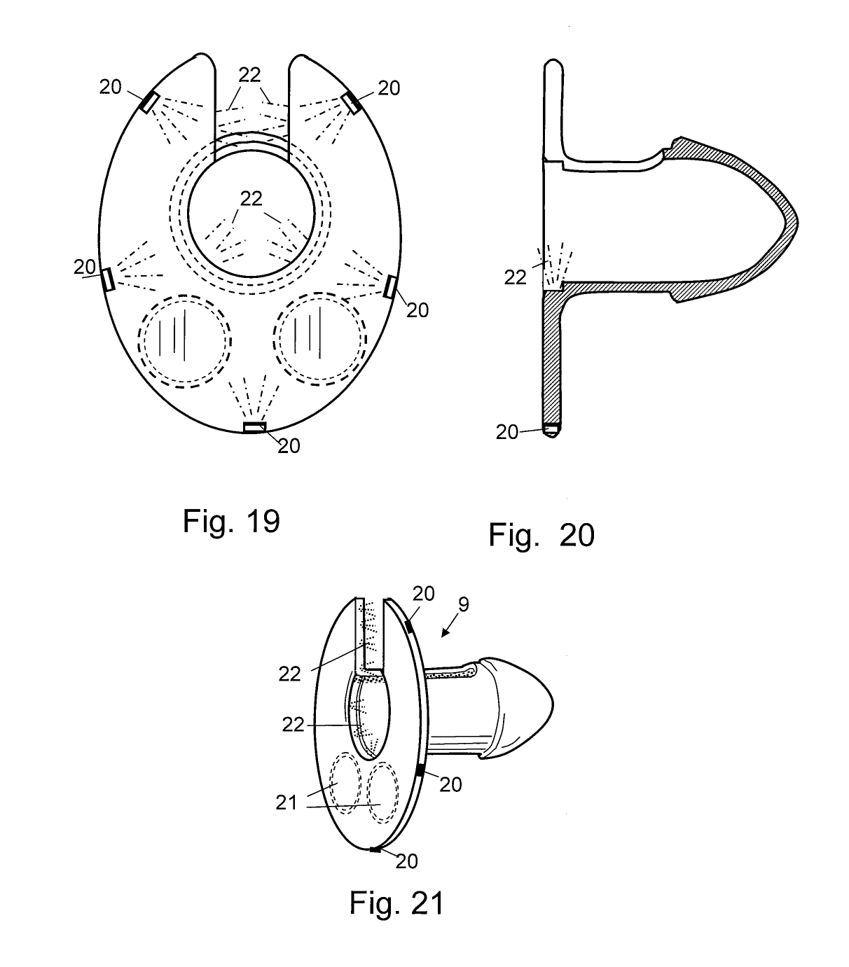

FIGS. 19, 20 and 21 show different views of another variation of an illumination system integrated in the labia spreader 9. In this implementation, the material in the labia spreader 9 is translucent and has an additional purpose of guiding light from light sources to direct it onto the urethral meatus. For this embodiment, one or more LEDs 20 direct light into the translucent material of labia spreader 9, and light rays 22 emerge from inwards facing areas of the labia spreader 9 in the direction to the urethral meatus. Batteries 21 may be integrated also in this embodiment of the labia spreader 9.

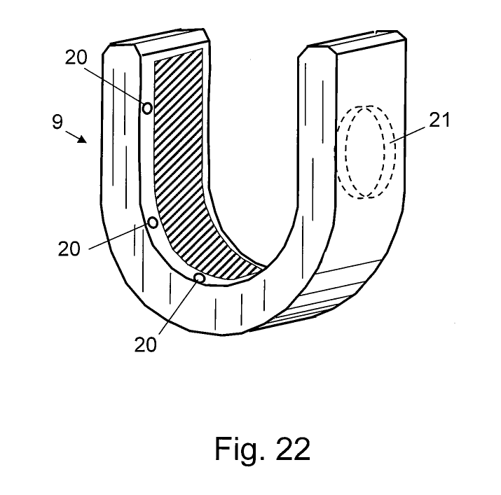

Now going to FIG. 22, this shows a U-shaped labia spreader 9 that was described previously in FIGS. 14-15, but here equipped with one or more light sources 20 that illuminate the urethral meatus. Batteries 21 may be easily integrated in also this embodiment of the labia spreader 9.

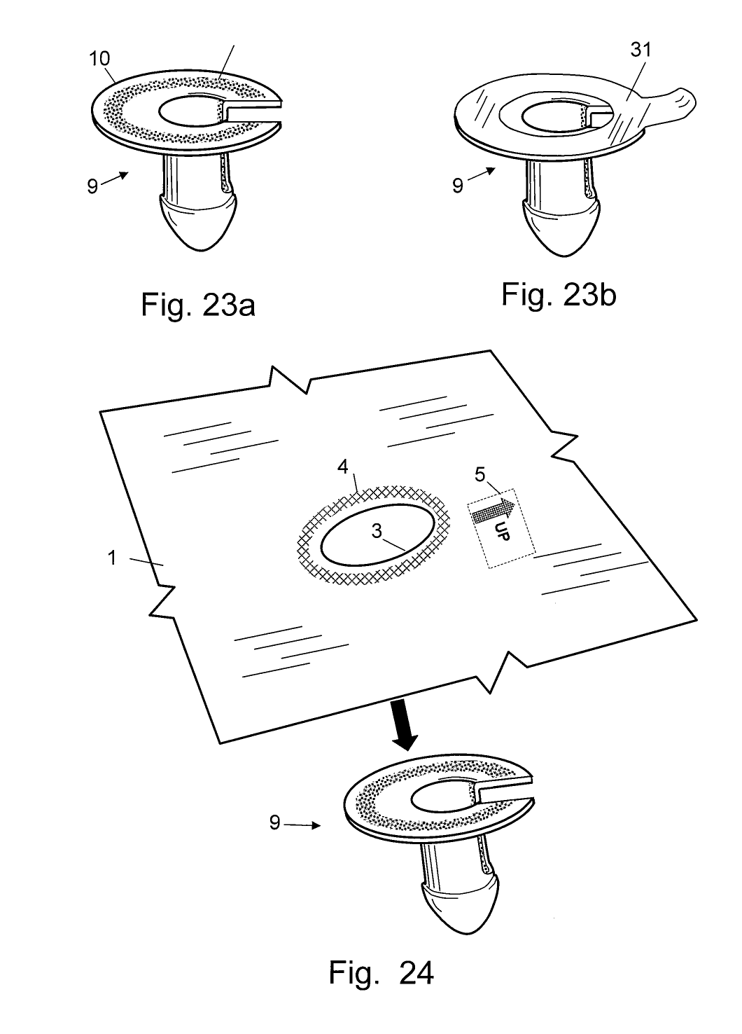

In FIG. 23a is shown a self adhesive coating 30 on the flange 10 of the labia spreader 9. The purpose of this adhesive is to attach the drape to the flange to create a sealed barrier isolating the urethral meatus from other patient areas. In FIG. 23b is shown a removable cover 31 that is used to protect the adhesive on the labia spreader 9 up to the time when the drape will be attached. It is obvious that the location of the adhesive coating may alternatively be on the drape portion of this drape system instead of the flange 10. It is also within the spirit of this invention to use other means of attachment of the drape, for instance magnetic materials or permanent attachment from the factory. However, the drape system application on a patient may be easier by first inserting only the labia spreader and then attaching the drape.

FIG. 24 shows how the drape 1 is attached to the labia spreader 9. The alignment marking 4 and the aperture marking 4 are used to properly mount the drape 1 to the labia spreader 9. This procedure is preferably done after the labia spreader 9 is inserted into the patient, but a pre-assembled drape system is also possible.

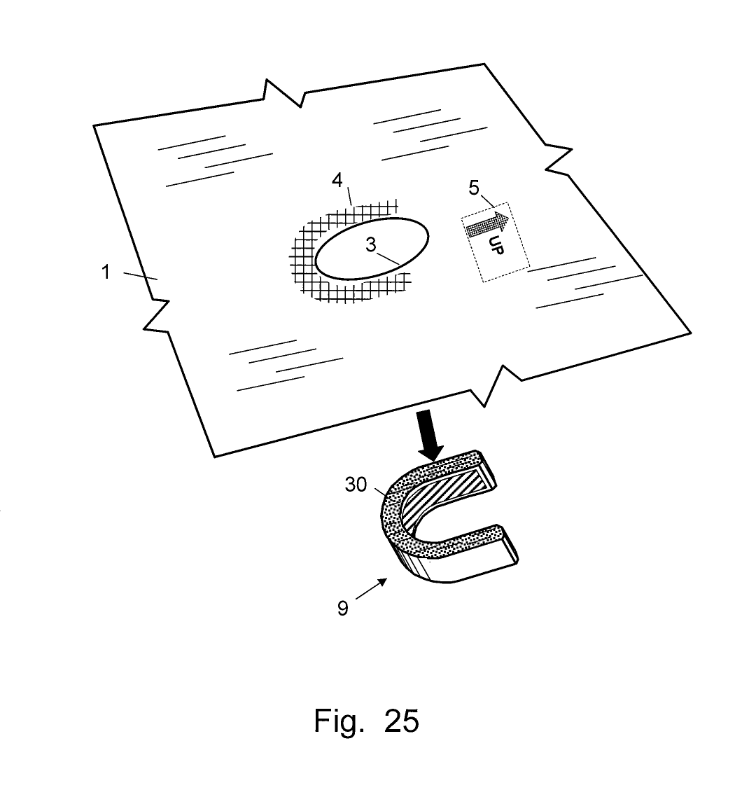

FIG. 25 shows how the previously mentioned U-shaped version of the labia spreader can be mounted via the self adhesive layer 30 applied on its flat side. The opening in the U-shape will be facing the urethral meatus side of the patient.

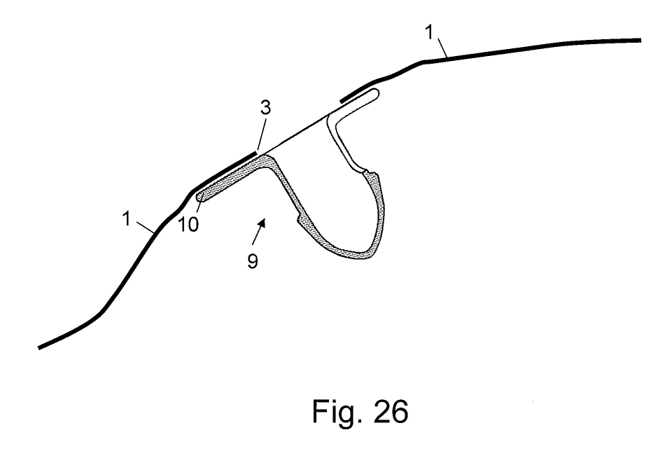

FIG. 26 shows how the drape 1 attaches and seals to flange 10 of labia spreader 9. This procedure is preferably done after the labia spreader is inserted into the patient.

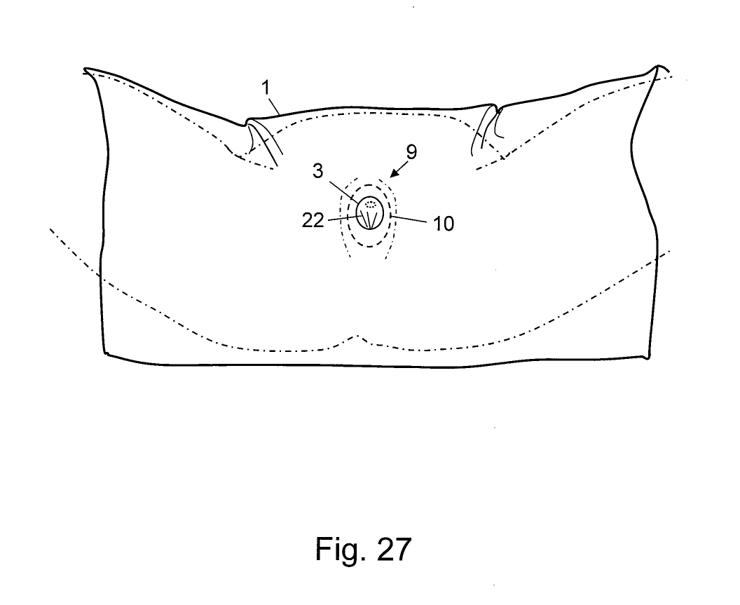

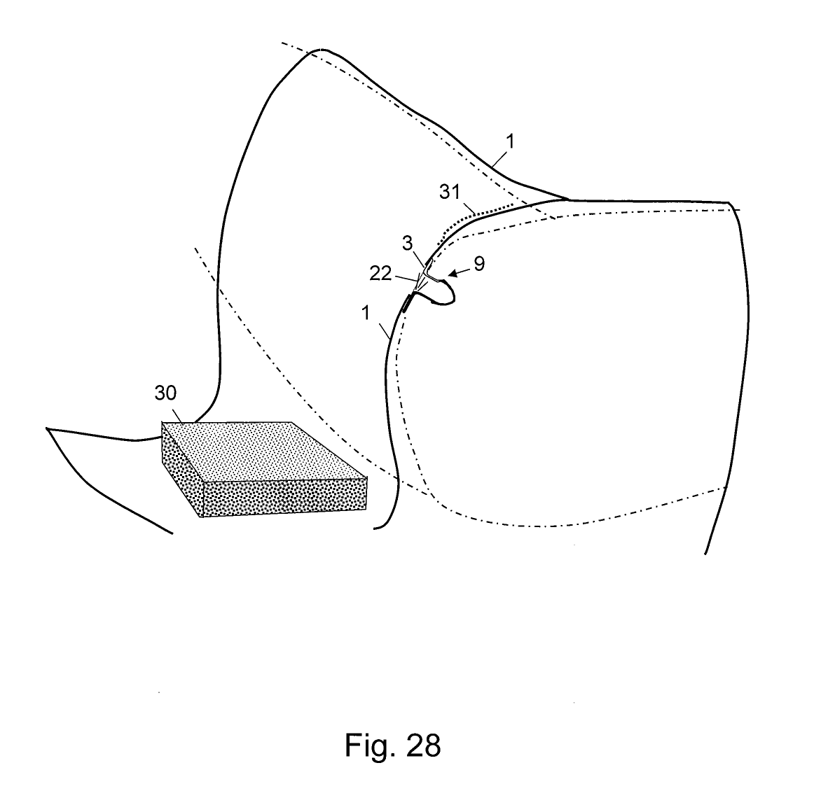

FIGS. 27 and 28 show different views of the complete drape system utilized on a schematically drawn female patient that is now ready for a urology procedure.

In FIG. 27 is illustrated the operator's typical view of a female patient with the invention drape system in place. It is remarked that only a very small area of the patient is exposed in the aperture 3 of drape 1 for the procedure compared to prior art, since only the urethral meatus area with nearby tissue areas, while this exposed area is completely surrounded by the sterile side of the drape 1. The drape 1 is attached and sealed to flange 10 of labia spreader 9. If this figure is compared with FIGS. 1 and 2 for prior art, the difference is clearly observed. This greatly reduces the risk for procedure complication by contamination from other patient areas. Furthermore, the drape 1 stays in place close to the patient by the attachment and sealing of drape 1 to labia spreader 9. This assures that the drape is not in the way for the operator and the aperture 3 is always exposing the urethral meatus.

Additionally in FIG. 27, is schematically illustrated the optional illumination feature of this drape system. Light rays 22 are emitted from light sources in the labia spreader 9 towards the urethral meatus on the patient, thus making this area clearly visible for the operator for safe insertion of urological instruments like catheters or endoscopes.

Now referring to FIG. 28, this shows a schematic vertical cross section of the drape system applied on a female patient. Drape 1 may continuously cover the entire genital area, as well as upper part of the patient's legs and lower part of her torso. The aperture 3 provides access to the urethral meatus through the openings of labial spreader 9. The illumination devices in labial spreader 9 emit light rays 22 towards the meatus area. It is noted that in typical female patient positions for urology procedures, normal medical room illumination from the ceiling will not easily reach the meatus area.

In FIG. 28 is also visualized an improved urology procedure liquid spill management system that may be incorporated because of the unique features in this drape system. As an effect of the sealed attachment of the drape 1 to the labia spreader 9, liquids emerging from meatus of the female patient or from urology instruments or irrigation, will stay on the operator side of the drape. Such liquids may be collected by one or more absorber pads 30, preferably using superabsorbent materials and gelling agents that make the absorber pad 30 non dripping semi solid disposable and eliminates medical waste liquid collection and handling. Optionally, a foldable flap 31 of preferably thin clear material, may be attached to a top area of the drape 1, close to the torso of the patient, for permitting easy urethral meatus access when folded up, and when folded down it will reduce risk for liquid splashing on the operator or on equipment.

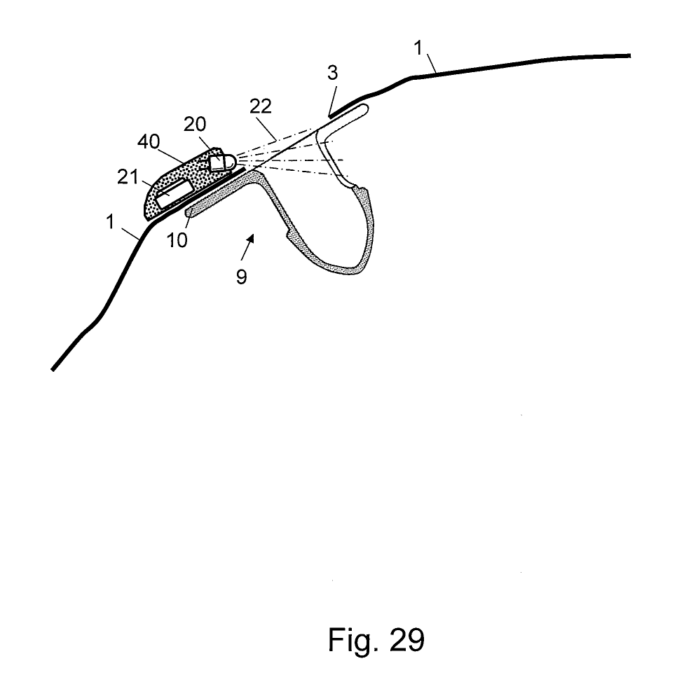

In FIG. 29 is shown another embodiment with an illumination module 40 attached to the outside of drape 1 to deliver light to the patient through the aperture 3. The illumination module 40 contains one or more light sources 20, for instance LED's, and a battery 21. The light source 20 is oriented to illuminate the patient's urethral meatus by the emitted light 22. This embodiment can permit illumination of the meatus with or without the use of the labia spreader 9. However, if the labia spreader 9 is not present, the procedure requires manual labia separation for access, and this would also degrade the antimicrobial and liquid handling integrity. Module 40 is adhesively attached to drape 1 or is attached thereto just prior to use of the inventive system.

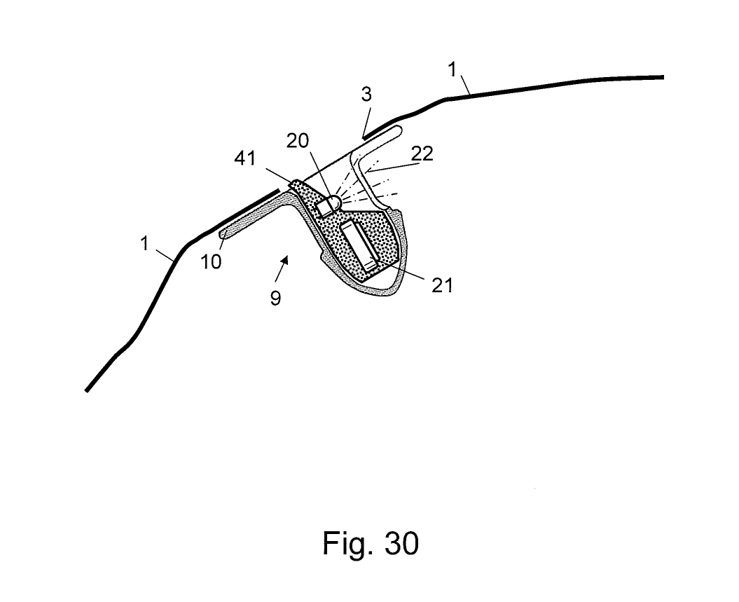

In FIG. 30 is shown yet another embodiment with a plug-in illumination module 41 that is insertable into the labia spreader 9. The plug-in illumination module 41 contains one or more light sources 20, for instance LED's, and a battery 21. The light source 20 is oriented to illuminate the patient's urethral meatus by the emitted light 22. This embodiment permits a choice of different type of illumination devices for a procedure but it adds one more module to be handled at the surgical procedure, and may slightly obscure urethral meatus access. Module 41 can be adhesively attached to spreading device 9 prior to use or at the time of use after the spreading device has been inserted into the vagina.

In conclusion, this novel surgical drape system for female patients provides significant advances in the art for safer and more predictable urology procedures It contains a number of features that may be used in a combination for maximum benefit in most cases, but features may also be used individually to provide specific improvements, as required from a case by case basis.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

1-48. (canceled)

49. A system for a urological medical procedure for providing a sterile environment and illumination of the female urethral meatus during isolated access of the urethral meatus of a female patient during a urological procedure, comprising a labia spreading device allowing access to the urethral meatus of the patient through an opening; a sterile, flat, planar, flexible drape adapted to be placed over said device to provide an upper sterile environment, said drape including an aperture to be located over the urethral meatus access opening of said device; and a light source integral with the device or drape, and positioned for directly illuminating the urethral meatus of the patient when the system is in use, said light source being carried on the exterior of the device or drape or being carried in the interior of the device or drape.

50. The system of claim 49 wherein the light source is carried on the exterior of the device or drape.

51. The system of claim 49 wherein the light source is carried in the interior of the device or drape.

52. The system of claim 51 wherein the device or drape carrying the light source is translucent or transparent at least in its area where illumination from the light source is focused onto the urethral meatus.

53. The system of claim 49 wherein the light source is a light emitting diode (LED).

54. The system of claim 49 wherein the light source is adhesively attached to the device or drape.

55. The system of claim 49 wherein the light source is attached by Velcro®-type fastener to the device or drape.

56. The system of claim 49 wherein indicia on said drape locates the position of the aperture.

57. The system of claim 49 wherein the device and the drape are attached to each other.

58. The system of claim 49 wherein the device has an upper flat surface allowing isolated access to the urethral meatus of the patient through an opening.

59. The system of claim 58 wherein the device and the drape are adhesively attached to each other.

60. The system of claim 56 wherein the drape includes a splittable tear line extending from a drape edge to the aperture.

61. The system of claim 49 wherein the device comprises a substantially flat body having opposed external side walls set at a distance from each other for spreading the labia, the opposed side walls having a depth sufficient for engaging the labia, a vaginal plug integral with and extending outwardly from a bottom surface of the body, the body including an opening through which the urethral meatus can be accessed when the vaginal plug has been inserted into the vagina of the patient.

62. The device of claim 61 wherein the light source is located within the vaginal plug.

63. The system of claim 61 in which the vaginal plug comprises a shaft attached to and extending substantially perpendicular from the body, the shaft terminating in an insertable enlarged head.

64. The system of claim 63 wherein the shaft terminates in an insertable bulbous head.

65. The system of claim 61 in which the shaft has an annular cross-section.

66. The system of claim 61 in which the vaginal plug is impervious to body fluids.

67. The system of claim 65 in which the meatus access opening is formed between an outer radius of the shaft and the periphery of the body.

68. The system of claim 67 wherein the meatus access opening is a slot which extends through the periphery of the body.

69. The system of claim 61 in which the opposed external side walls of the device are inwardly compressible and then releasable substantially to their original location for engaging the labia.

70. The system of claim 61 in which the body of the device contains a flat surface opposite to the vaginal plug, and having an inwardly stepped flange thereon which extends outwardly from said surface for extending beyond the spread labia.

71. The system of claim 69 in which the body of the device contains a flat surface opposite to the vaginal plug, and having an inwardly stepped flange thereon which extends outwardly from said surface for extending beyond the spread labia.

72. The system of claim 70 in which the device includes a second slot in the body opposite to the slot of claim 69.

73. The system of claim 72 in which the opposed external side walls of the device are inwardly compressible and then releasable substantially to their original location for engaging the labia.

74. The system of claim 61 wherein the flat body contains at least one integrated light source which illuminates the opening through which the urethral meatus is accessed.

75. The system of claim 74 wherein the flat body contains a plurality of integrated light sources which illuminate the opening through which the urethral meatus is accessed.

76. The system of claim 49 in which the device comprises a U-shaped body forming opposed external side walls and being substantially flat on one side and with its external side walls set at a distance apart from one another for spreading the labia, the opposed side walls having a depth sufficient for engaging the labia, the external side walls being inwardly compressible and then releasable substantially to their original location for engaging the labia.

77. The system of claim 76, wherein the device includes at least one integrated light source set in a side wall for illuminating the urethral meatus.

78. The system of claim 76 in wherein the device includes a plurality of integrated light sources set in the side walls for illuminating the urethral meatus.

79. The system of claim 61 in which the device is formed of a flexible material or contains a spring.

80. The system of claim 49 wherein the device comprises a substantially flat body having opposed external side walls set at a distance from each other for spreading the labia, the opposed side walls having a depth sufficient for engaging the labia, a balloon extending outwardly from a bottom surface of the body, said balloon functioning as a vaginal plug when inflated, the body including an opening through which the urethral meatus can be accessed when the balloon has been inserted into the vagina of the patient.

81. A system for use in carrying out a urological procedure on a human female patient comprising the system of claim 80 and a means for inflating the balloon.

82. The system of claim 80 wherein the opposed external side walls are inwardly compressible and then releasable to substantially their original location for engaging the labia.

83. The system of claim 49 wherein the device or the drape carries an adhesive coating on a portion of an upper surface of the device and/or on a lower surface of the drape.

84. The system of claim 83 in which the adhesive coating is covered by a removable cover.

85. The system of claim 49 wherein the device and the drape are attached to each other prior to use.

86. A device for spreading the labia of a human female patient and providing access to the urethral meatus of the patient during a urological procedure which comprises a substantially flat body having opposed external side walls set at a distance from each other for spreading the labia, the opposed side walls having a depth sufficient for engaging the labia, a balloon extending outwardly from a bottom surface of the body, said balloon functioning as a vaginal plug when inflated, the body including an opening through which the urethral meatus can be accessed when the balloon has been inserted into the vagina of the patient, said device containing an integral light source positioned for directly illuminating the urethral meatus of the patient when the device is in use during a urological procedure, said light source being carried on the exterior of the device or being carried within the interior of the device.

87. A device for spreading the labia of a human female patient and providing access to the urethral meatus of the patient during a urological procedure which comprises a substantially flat body having opposed external side walls set at a distance from each other for spreading the labia, the opposed side walls having a depth sufficient for engaging the labia, a vaginal plug integral with and extending outwardly from a bottom surface of the body, the body including an opening through which the urethral meatus can be accessed when the vaginal plug has been inserted into the vagina of the patient, and wherein said body has on a upper surface having an inwardly stepped flange thereon which extends outwardly from said surface for extending beyond the spread labia, said device containing an integral light source positioned for directly illuminating the urethral meatus of the patient when the device is in use during a urological procedure, said light source being carried on the exterior of the device or being carried within the interior of the device.

88. A device for spreading the labia of a human female patient and providing access to the urethral meatus of the patient during a urological procedure which comprises a substantially flat body having opposed external side walls set at a distance from each other for spreading the labia, the opposed side walls having a depth sufficient for engaging the labia, a vaginal plug integral with and extending outwardly from a bottom surface of the body, the body including a first slot opening to the periphery of the body and through which the urethra meatus can be accessed when the vaginal plug has been inserted into the vagina of the patient, and including a second slot in the body opposite to the first slot, said device containing an integral light source positioned for directly illuminating the urethral meatus of the patient when the device is in use during a urological procedure, said light source being carried on the exterior of the device or being carried within the interior of the device.

89. The device of claim 85 wherein opposed external side walls are inwardly compressible and then releasable substantially to their original location for engaging the labia.

90. A medical method for providing a sterile environment during isolated access of the urethral meatus of a female patient during a urological procedure which comprises (1) spreading the labia of the patient using a labia spreader having an upper flat surface and an opening allowing access to the urethral meatus of the patient; (2) covering the labia spreader and surrounding area of the patient with a sterile, flat, planar, flexible drape which lays flat over the upper surface of the labia spreader and which has when laid in the flat position an aperture located over the urethral meatus opening of the labia spreader and (3) directly illuminating the urethral meatus of the patient with a light source integral with and carried on or within said labia spreader or integral with and carried on or within said drape.

91. The method of claim 90 in which a portion of the upper flat surface of the labia spreader carries an adhesive covered by a removable cover, and after positioning the labia spreader on the patient in the spread position, the cover is removed to expose the adhesive, after which the drape is adhesively sealed to the upper surface of the labia spreader.

92. The method of claim 90 in which a portion of the lower surface of the drape carries an adhesive covered by a removable cover, and after positioning the labia spreader on the patient in the spread position, the cover is removed to expose the adhesive, after which the drape is adhesively sealed to the upper surface of the labia spreader.

93. The method of claim 90 including the use of a liquid absorbing pad on an exterior side of the drape and wherein a liquid flow controlling foldable flap is attached to a top area of the drape closest to a torso of the patient for directing liquid to the absorbing pad.
